(19) United States
(12) Patent Application Publication
(10) Pub. No.: US 2024/0117396 A1
(43) Pub. Date: Apr. 11, 2024

(54) Bacillus Licheniformis Cell Stably and Repeatedly Used for Conversion and Synthesis of D-psicose

(71) Applicant: WUXI INSTITUTE FOR SPECIALIZED NUTRITIONAND HEALTH CO., LTD, Wuxi (CN)

(72) Inventors: Youran LI, Wuxi (CN); Zhiyong Wu, Wuxi (CN); Guiyang Shi, Wuxi (CN)

(21) Appl. No.: 18/539,751

(22) Filed: Dec. 14, 2023

Related U.S. Application Data
(63) Continuation of application No. PCT/CN2023/118453, filed on Sep. 13, 2023.

(30) Foreign Application Priority Data
Sep. 26, 2022 (CN) 2022111762183

(51) Int. Cl. C12P 19/02; C12N 1/02; C12N 1/20

(52) U.S. Cl. CPC C12P 19/02; C12N 1/02; C12N 1/205; C12R 2001/10

(57) ABSTRACT

The present disclosure discloses a Bacillus licheniformis cell stably and repeatedly used for the conversion and synthesis of D-psicose, and belongs to the field of biotechnology. D-psicose is a functional sugar with high health value and has broad application prospects in food, medicine, health care and other fields. The B. licheniformis cell of the present disclosure efficiently expresses recombinant D-psicose-3-epimerase, and can convert high-concentration fructose as a substrate into D-psicose. After the whole-cell catalyst is reused 10 times, the conversion rate is not less than 30%. Compared with pure enzyme or single-use whole-cell conversion methods, this technology for the synthesis of D-psicose has significant advantages such as low catalyst cost and simple separation and purification.

### REFERENCE TO SEQUENCE LISTING

The instant application contains a Sequence Listing in XML format as a file named “YGHY-2023-03-SEQ.xml”, created on Nov. 30, 2023, of 14.1 kB in size, and which is hereby incorporated by reference in its entirety.

### TECHNICAL FIELD

The present disclosure relates to a Bacillus licheniformis cell stably and repeatedly used for the conversion and synthesis of D-psicose, and belongs to the field of biotechnology.

### BACKGROUND

D-psicose is a sugar existed in nature but in very small amounts. It has a sweetness reaching 70% of sucrose, and has a refreshing taste without bitterness. Furthermore, D-psicose cannot be metabolized or is rarely metabolized in the human body. It is evaluated as the most potential sucrose substitute and has broad application prospects in food, medicine, health care and other fields.

The synthesis of D-psicose through chemical routes is difficult and inefficient. The bioconversion method has the advantages of mild reaction conditions, few by-products, simple purification steps, and environmental friendliness, and has gradually become the main direction for the synthesis of D-psicose. At present, the bioconversion of D-psicose mainly relies on the expression of D-psicose-3-epimerase in hosts such as Escherichia coli, Corynebacterium glutamicum, or Bacillus subtilis, then the acquisition of pure enzyme through cell disruption, and the conversion and synthesis of D-psicose using D-fructose as the substrate. In such production method, the pure enzyme as a catalyst can only be used once and brings challenges to the isolation of the product. At present, there are relatively few reports on the use of whole-cell conversion to synthesize D-psicose, and the main host cells used are E. coli, C. glutamicum, or B. subtilis. The whole-cell catalysis eliminates the need for cell disruption after fermentation, and the catalyst can be easily separated from the product by centrifugation or filtration. However, cells of E. coli, C. glutamicum or B. subtilis are easily lysed at a catalytic temperature of 60-70° C.; and even if the cells are recovered, they are easily infected with phages during the process. According to literature reports, B. subtilis suffers from severe autolysis phenomenon. As noted in “However, there is a problem with cell autolysis in fermentation cultures of B. subtilis.” “As mentioned in the article by Regamey and Karamat et al., after B. subtilis was subjected to thermal excitation at 50° C., prophage sp6 was induced, and many cells underwent autolysis.”, and also as pointed out in the paper Ren, K.; Wang, Q.; Hu, M.; Chen, Y.; Xing, R.; You, J.; Xu, M.; Zhang, X.; Rao, Z. Research Progress on the Effect of Autolysis to Bacillus subtilis Fermentation Bioprocess. Fermentation 2022, 8, 685., B. subtilis has the problem of autolysis, and especially in high temperature environment (50° C.), the autolysis is more severe. Therefore, it is difficult to reuse whole-cell catalysts.

B. licheniformis is widely used as a production host for food enzyme preparations and important nutritional chemicals. Its products are certified by the FDA as “generally regarded as safe” (GRAS) safety level. On the other hand, this strain is a typical heat-resistant microorganism that can grow at 50° C. The cultured cells have excellent stability at 60-70° C., and there are few problems with phage infection. The engineered bacteria constructed using B. licheniformis can be stably and repeatedly used for the conversion and synthesis of D-psicose, which has obvious advantages over existing methods in terms of production efficiency and cost.

### SUMMARY

The first objective of the present disclosure is to provide a gene encoding D-psicose-3-epimerase with a nucleotide sequence shown as SEQ ID NO. 1.

The second objective of the present disclosure is to provide a vector for expressing the above gene.

In one embodiment, the vector is pHY300-PLK.

The third objective of the present disclosure is to provide a cell carrying the above gene or the above vector.

In one embodiment, the cell is B. licheniformis.

In one embodiment, the B. licheniformis includes B. licheniformis CICIM B1341.

The fourth objective of the present disclosure is to provide recombinant B. licheniformis carrying the above gene encoding D-psicose-3-epimerase.

In one embodiment, the gene of D-psicose-3-epimerase is initially expressed from promoter P_{Ian}.

In one embodiment, the gene of D-psicose-3-epimerase is integrated into a site of amylase encoding gene amyL in a B. licheniformis genome.

In one embodiment, a nucleotide sequence of the promoter Plan is shown in SEQ ID NO. 2.

In one embodiment, a nucleotide sequence of the amylase encoding gene is shown in SEQ ID NO. 4.

In one embodiment, an expression vector of the recombinant B. licheniformis is pHY300-PLK.

In one embodiment, the recombinant B. licheniformis uses B. licheniformis CICIM B1341 as a host.

In one embodiment, the B. licheniformis CICIM B1341 is derived from a natural strain preserved in China University Industrial Microbiology Resource Platform.

The fifth objective of the present disclosure is to provide a whole-cell catalyst, which includes the above recombinant B. licheniformis.

The sixth objective of the present disclosure is to provide a method for constructing the recombinant B. licheniformis, including fusing the D-psicose-3-epimerase gene al with the promoter P_{Ian} and terminator ter to obtain an integrated fragment, after connecting the integrated fragment to the pHY300-PLK vector, obtaining recombinant plasmid pHY300-PIan-a1, transferring the recombinant plasmid into B. licheniformis, and integrating expression cassettes of the D-psicose-3-epimerase gene into the site of the amylase encoding gene amyL in the B. licheniformis genome through subculture.

In one embodiment, the nucleotide sequence of the promoter P lan is shown in SEQ ID NO. 2, and the nucleotide sequence of the terminator ter is shown in SEQ ID NO. 3.

In one embodiment, the nucleotide sequence of the amylase encoding gene is shown in SEQ ID NO. 4.

The seventh objective of the present disclosure is to provide a method for synthesis of D-psicose through whole-cell conversion, including using D-fructose as a substrate, and adding the recombinant B. licheniformis or wet cells of the whole-cell catalyst, reacting at 50-70° C. for 5-10 h.

In one embodiment, after the reaction is completed, the recombinant B. licheniformis or the whole-cell catalyst is recovered, reused, and added to a reaction system using the D-fructose as the substrate to synthesize D-psicose.

In one embodiment, the number of repeated uses is more than 10 times.

In one embodiment, the recombinant B. licheniformis or the whole-cell catalyst is inoculated into a seed medium and cultured at 37° C. to 42° C. for 12-24 h, then transferred to a fermentation medium at an inoculation amount of 1% to 5% by volume, and cultured at 37° C. to 42° C. for 24-36 h, and wet cells are collected.

In one embodiment, an OD₆₀₀ value of the wet cells is 50 to 100.

In one embodiment, the seed medium includes tryptone 8-12 g/L, yeast powder 4-6 g/L, and NaCl 8-12 g/L.

In one embodiment, the fermentation medium includes (g/L): 20-70 of sucrose, 10-30 of cottonseed protein, 9.12 of K₂HPO₄·3H₂O, 1.36 of KH₂PO₄, 10 of (NH₄)₂HPO₄; and initial pH is 7.5.

The present disclosure also provides application of the above gene, or the above vector, or the above cell, or the above recombinant B. licheniformis, or the application of the above whole-cell catalyst in the preparation of products containing D-psicose.

Compared with the related art, the positive progressive effects of the present disclosure are as follows:
1. The present disclosure uses the food-safe microorganism B. licheniformis as a starting strain, and utilizes its cell's good tolerance to high temperatures to protect the D-psicose-3-epimerase expressed in the cell during the catalytic process. The recombinant B. licheniformis provided by the present disclosure can proliferate at 37-42° C.
2. The present disclosure constructs the gene encoding D-psicose-3-epimerase with a nucleotide sequence as shown in SEQ ID NO. 1 into B. licheniformis to obtain the whole-cell catalyst. The whole-cell catalyst can repeat the fermentation reaction 10 times at a temperature of 60° C., and the conversion rate is not less than 30%. Compared with other pure enzyme conversion methods that use one-time catalysts or whole-cell catalysis methods that are prone to cell rupture, the technical solution of the present disclosure has significant advantages in terms of production cost and efficiency.

### BRIEF DESCRIPTION OF FIGURES

FIG. 1: Map and enzyme digestion verification of recombinant plasmid pHY300-P_{Ian}-a1.

FIG. 2: Map of recombinant plasmid pMA5-a1.

FIG. 3: HPLC spectrum of a reaction solution.

FIG. 4: Reusing 10 batches of whole-cell catalytic synthesis of D-psicose.

FIG. 5: Single ceramic membrane continuous bioconversion device.

### DETAILED DESCRIPTION

The present disclosure will be further described below with reference to the accompanying drawings and specific examples, but the examples do not limit the present disclosure in any form. Unless otherwise stated, the reagents and materials used in the following examples are commercially available or can be prepared by known methods.

Methods Involved in the Following Examples:
1. Determination method for D-psicose: A catalytic reaction system is centrifuged at 13000 r/min for 20 min. A supernatant is diluted 2 times with anhydrous ethanol, left to stand at 4° C. for 2 h, and then filtered with a 0.22 μM water membrane. D-psicose is detected by high performance liquid chromatography.

HPLC detection conditions: 250 mm*4.6 μm Polyamino HILIC column is used, a mobile phase is acetonitrile:water=75:25, a detector is a differential detector, a flow rate is 1 mL/min, a column temperature is 40° C., and a differential detector cell temperature is 40° C.
2. Enzyme activity determination method for D-psicose-3-epimerase: 1 mL of fermentation broth sample is taken, and centrifuged at 12000 r/min for 5 min. A precipitate is repeatedly washed and blown with phosphate buffer (50 mmol/L, pH 7.5), and centrifuged three times. The bacterial precipitate is diluted to an appropriate concentration, and resuspended with a fructose substrate solution prepared with HEPEs buffer (50 mmol/L, pH 7.5) to a final volume of 1 mL (fructose mass concentration of 90 g/L, and Co²⁺ and Mn²⁺ concentration of 1 mmol/L). After incubation at 60° C. for 10 min and boiling to inactivate enzyme for 5 min, the resulting solution is centrifuged at 12000 r/min for 5 min. A supernatant is filtered through a 0.22 μm filter, and D-psicose is analyzed using HPLC conditions.

Definition of enzyme activity: the enzyme activity that converts fructose into 1 mg D-psicose within 1 h, which is defined as 1 U.

Plasmids and Strains Involved in the Following Examples:

B. licheniformis: CICIM B1341, preserved in this laboratory, can be purchased from the China University Industrial Microbiology Resource and Information Center of Jiangnan University.

Media Involved in the Following Examples:

Seed medium (g/L): 10 of tryptone, 5 of yeast powder, and 10 of NaCl.

Fermentation medium (g/L): 70 of sucrose, 30 of cottonseed protein, 9.12 of K₂HPO₄·3H₂O 9.12, 1.36 of KH₂PO₄ 1.36, 10 of (NH₄)₂HPO₄ 10; and initial is pH 7.5.

### Example 1: Construction of a Recombinant B. licheniformis Strain Overexpressing D-psicose-3-epimerase Gene

(1) Knockout of Amylase Encoding Gene amyL and Integrative Expression of D-Psicose-3-Epimerase Gene

Using a codon-optimized al gene fragment expression cassette fused with promoter and terminator as a template, the gene was amplified to obtain an expression cassette containing the al gene fragment with a nucleotide sequence as shown in SEQ ID NO. 1. Amplification primers were as follows:

Upstream primer:
SEQ ID NO. 5
5-GCGCGGATCCATGAAGCACGGTATCTATTA-3 (BamHI),;
Downstream primer:
SEQ ID NO. 6
5-CCGGAAGCTTGGAGTGTTTGTGACATTCTA-3 (HindIII),;

and

PCR conditions: denaturation at 94° C. for 2 min, denaturation at 98° C. for 30 s, annealing at 50° C. for 30 s, and extension at 68° C. for 1 min.

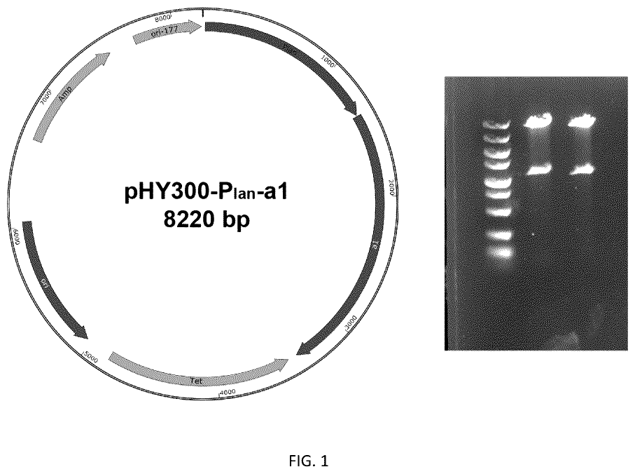

A pHY300-PLK vector (from the same source as Li, Y.; Jin, K.; Zhang, L.; Ding, Z.; Gu, Z.; Shi, G. Development of an Inducible Secretory Expression System in Bacillus licheniformis Based on an Engineered Xylose Operon. Journal of Agricultural and Food Chemistry 2018, 66, 9456-9464.) and the expression cassette containing the al gene fragment obtained by PCR amplification were digested with two restriction enzymes, BamHI and HindIII at 37° C., and ligated with T4 ligase at 16° C. The resulting ligation product was transformed into E. coli DH5a and screened using Ampicillin-resistant seed medium plates. Transformants were selected for plasmid extraction, enzyme digestion verification, and gene sequencing. Recombinant plasmid with correct sequencing was named pHY300-P_{Ian}-a1, as shown in FIG. 1.

The recombinant plasmid pHY300-P_{Ian}-a1 was extracted from E. coli, with addition of the homology arm of the amylase encoding gene amyL according to a method in Li, Y.; Jin, K.; Zhang, L.; Ding, Z.; Gu, Z.; Shi, G. Development of an Inducible Secretory Expression System in Bacillus licheniformis Based on an Engineered Xylose Operon. Journal of Agricultural and Food Chemistry 2018, 66, 9456-9464., and then introduced into B. licheniformis to obtain recombinant B. licheniformis BLA1 with integration of the al gene fragment expression cassette at a site of the amylase encoding gene amyL.

(2) Free Expression of D-Psicose-3-Epimerase Gene

Using a codon-optimized al gene fragment expression cassette fused with promoter and terminator as a template, the gene was amplified to obtain an expression cassette containing the al gene fragment with a nucleotide sequence as shown in SEQ ID NO. 1. Amplification primers were as follows:

Upstream primer:
SEQ ID NO. 5
5-GCGCGGATCCATGAAGCACGGTATCTATTA-3 (BamHI),;
Downstream primer:
SEQ ID NO. 6
5-CCGGAAGCTTGGAGTGTTTGTGACATTCTA-3 (HindIII),;

and

PCR conditions: denaturation at 94° C. for 2 min, denaturation at 98° C. for 30 s, annealing at 50° C. for 30 s, and extension at 68° C. for 1 min.

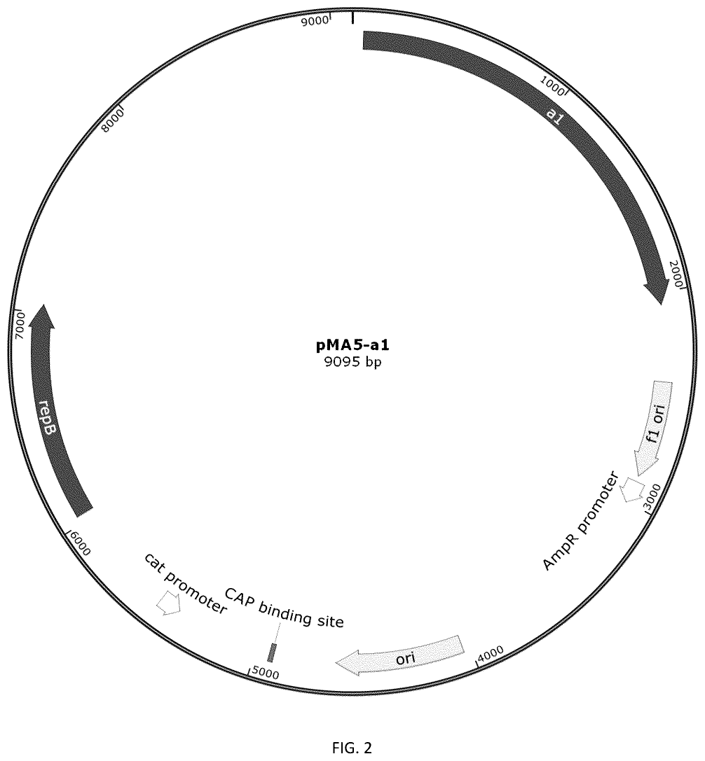

A pMA5 vector (from Xu, Yinbiao, et al. “Unraveling the specific regulation of the shikimate pathway for tyrosine accumulation in Bacillus licheniformis.” Journal of Industrial Microbiology and Biotechnology 46.8 (2019): 1047-1059.) and the expression cassette containing the al gene fragment obtained by PCR amplification were digested with two restriction enzymes, BamHI and HindIII at 37° C., and ligated with T4 ligase at 16° C. The resulting ligation product was transformed into E. coli DH5a and screened using Ampicillin-resistant seed medium plates. Transformants were selected for plasmid extraction, enzyme digestion verification, and gene sequencing. Recombinant plasmid with correct sequencing was named pMA5-a1, as shown in FIG. 2.

The recombinant plasmid pMA5-a1 was extracted from E. coli, and according to a method in Li, Y.; Jin, K.; Zhang, L.; Ding, Z.; Gu, Z.; Shi, G. Development of an Inducible Secretory Expression System in Bacillus licheniformis Based on an Engineered Xylose Operon. Journal of Agricultural and Food Chemistry 2018, 66, 9456-9464., introduced into B. licheniformis to obtain recombinant B. licheniformis BLA2 with free expression of the al gene fragment expression cassette.

(3) Knockout of Amylase Encoding Gene aprE and Integrative Expression of D-Psicose-3-Epimerase Gene

Using a codon-optimized al gene fragment expression cassette fused with promoter and terminator as a template, the gene was amplified to obtain an expression cassette containing the al gene fragment with a nucleotide sequence as shown in SEQ ID NO. 1. Amplification primers were as follows:

Upstream primer:
SEQ ID NO. 5
5-GCGCGGATCCATGAAGCACGGTATCTATTA-3 (BamHI),;
Downstream primer:
SEQ ID NO. 6
5-CCGGAAGCTTGGAGTGTTTGTGACATTCTA-3 (HindIII),;

and

PCR conditions: denaturation at 94° C. for 2 min, denaturation at 98° C. for 30 s, annealing at 50° C. for 30 s, and extension at 68° C. for 1 min.

A pHY300-PLK vector (from the same source as Li, Y.; Jin, K.; Zhang, L.; Ding, Z.; Gu, Z.; Shi, G. Development of an Inducible Secretory Expression System in Bacillus licheniformis Based on an Engineered Xylose Operon. Journal of Agricultural and Food Chemistry 2018, 66, 9456-9464.) and the expression cassette containing the al gene fragment obtained by PCR amplification were digested with two restriction enzymes, BamHI and HindIII at 37° C., and ligated with T4 ligase at 16° C. The resulting ligation product was transformed into E. coli DH5a and screened using Ampicillin-resistant seed medium plates. Transformants were selected for plasmid extraction, enzyme digestion verification, and gene sequencing. Recombinant plasmid with correct sequencing was named pHY300-P_{Ian}-a1, as shown in FIG. 1.

The recombinant plasmid pHY300-P_{Ian}-a1 was extracted from E. coli, with addition of the homology arm of the amylase encoding gene aprE according to a method in Li, Y.; Jin, K.; Zhang, L.; Ding, Z.; Gu, Z.; Shi, G. Development of an Inducible Secretory Expression System in Bacillus licheniformis Based on an Engineered Xylose Operon. Journal of Agricultural and Food Chemistry 2018, 66, 9456-9464., and then introduced into B. licheniformis to obtain recombinant B. licheniformis BLA3 with integration of the al gene fragment expression cassette at a site of the amylase encoding gene aprE.

### Example 2: Culture and Enzyme Production of Different Recombinant Bacterial Cells

The recombinant B. licheniformis BLA1, BLA2 and BLA3 constructed in Example 1 were respectively inoculated into fermentation media and cultured at 37° C. Samples were taken at 72 h to detect the cell OD 600 and product concentration. Table 1 showed OD 600 values and enzyme activity yields of the cells after fermentation culture of different recombinant bacteria. The results showed that the enzyme activity of integrative expression of the D-psicose-3-epimerase gene into the site of the amylase encoding gene amyL was significantly higher than the enzyme activity of free expression or integrative expression into the site of the amylase encoding gene aprE.

TABLE 1
Cell concentration and enzyme activity of recombinant
bacteria at different temperatures

| | | Enzyme activity |
|---|---|---|
| | OD₆₀₀ | (U/mL) |
| BLA1 | 31.44 | 106.25 |
| BLA2 | 28.21 | 59.30 |
| BLA3 | 33.83 | 52.65 |

### Example 3: Culture and Enzyme Production of Recombinant Bacterial Cells BLA1 at Different Temperatures

The recombinant B. licheniformis BLA1 constructed in Example 1 was inoculated into a fermentation medium and cultured at 37° C. and 42° C. respectively. On the third and fourth days, samples were taken to detect the cell OD 600 and product concentration. Table 2 showed OD₆₀₀ values of bacterial cells at 37° C. and 42° C. The number of cells was higher at 37° C. after 72 h of fermentation culture, and the number of cells was higher at 42° C. after 96 h of fermentation culture. At the same temperature, the number of bacterial cells on the fourth day was higher than that on the third day, indicating that the growth trends of the two were the same, but the growth rate of cell mass was different. The growth rate of cell mass was higher at 42° C. The enzyme activity was higher at 37° C. after 72 h of fermentation culture.

TABLE 2
Cell concentration and enzyme activity of recombinant
bacteria at different temperatures

| | OD₆₀₀ at | OD₆₀₀ at | Enzyme activity at | Enzyme activity at |
|---|---|---|---|---|
| | 37° C. | 42° C. | 37° C. (U/mL) | 42° C. (U/mL) |
| 72 h | 31.44 | 20.48 | 106.25 | 45.00 |
| 96 h | 40.10 | 54.00 | 77.75 | 44.75 |

### Example 4: Reusable Whole-Cell Conversion

(1) Cell Filtration Through Centrifugation

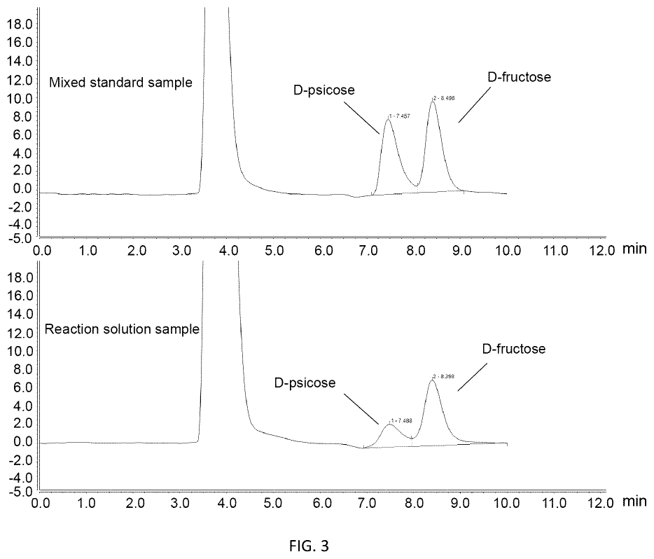

The recombinant B. licheniformis BLA1 constructed in Example 1 was inoculated into a seed medium and cultured at 37° C. for 12-24 h, and then transferred to a fermentation medium at an inoculation amount of 1% to 5% (v/v) and cultured at 37° C. for 24-36 h. 1 mL of fermentation broth was taken, the cell OD₆₀₀ value was measured using 100 μl of the broth, and the remaining fermentation broth was centrifuged at 12000 r/min and 4° C. for 20 min. A supernatant was discarded, and a precipitate was washed once with 10 mM PB buffer. Centrifugation was carried out again. A supernatant was discarded, and a corresponding volume of PB buffer was added to resuspend a precipitate so as to achieve the cell OD 600 value of 80. An appropriate amount of cell suspension was taken and added to 500 mM HEPEs buffer with pH 7.5 (containing 5 mM CoCl₂ and 5 mM MnCl₂) and an equal volume of 400 g/L (initial concentration) fructose solution, where the volume ratio of fructose solution and HEPEs buffer was 10:3, and the reaction was carried out in a metal bath at 60° C. for 6 h. After the reaction is completed, the mixture was centrifuged at 12,000 r/min for 20 min, and a precipitate was used for the reuse of the whole-cell catalyst. A supernatant was mixed with an equal volume of anhydrous ethanol, left to stand at 4° C. for 2 h or more, and centrifuged at 12,000 r/min for 20 min. A supernatant was taken and diluted 50 times, filtered through a water-based filter head to 200 μL in a lined tube, and placed in a liquid phase detection bottle for liquid phase detection. The results were shown in FIG. 3: There was an analytical peak of D-psicose in the reaction solution, the yield of D-psicose was 124.4 g/L, and the conversion rate was 31.4%.

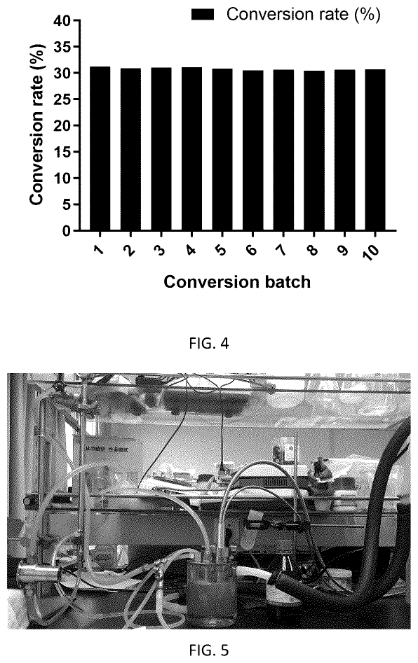

The same batch of whole-cell catalyst was reused 10 times. The results showed that after the whole-cell catalyst was reused 10 times, the conversion rate was no less than 30% (FIG. 4).

(2) Cell Filtration Through Microfiltration

To achieve continuous production of D-psicose using B. licheniformis, a small-scale continuous bioconversion device was assembled through coupling ceramic membranes, pumps, constant temperature water baths, reactors and other equipment (FIG. 5), aiming to simulate industrial production. The selection of the circulation unit was based on industrial production applications. The pressure on cells during the production and preparation process was greater than or equal to the industrial production level.

The stability and catalytic activity of bacterial cells for continuous D-psicose production were tested through ceramic membrane microfiltration cycles. A total of 10 microfiltrations were completed.

TABLE 3
Cell stability test and continuous microfiltration data

| | | Unit cell | | Membrane | |
|---|---|---|---|---|---|
| | | enzyme | Flow | filtration | Microfiltration |
| Sample | | activity | rate | pressure | time |
| No. | OD₆₀₀ | U/OD₆₀₀ | (L/h) | (kg/cm²) | (min) |
| F0 | 3.0 | — | 150 | 0.9 | — |
| F1 | 3.0 | 3.4 | 150 | 0.9 | 33 |
| F2 | 3.0 | 3.4 | 150 | 0.9 | 50 |
| F3 | 3.0 | 3.4 | 150 | 0.9 | 53 |
| F4 | 3.0 | 3.3 | 150 | 0.9 | 57 |
| F5) | 3.0 | 3.3 | 150 | 0.9 | 67 |
| F6 | 2.9 | 3.3 | 150 | 0.9 | 65 |
| F7 | 2.9 | 3.3 | 150 | 0.9 | 62 |
| F8 | 2.9 | 3.3 | 150 | 0.9 | 62 |
| F9 | 2.9 | 3.3 | 150 | 0.9 | 62 |
| F10 | 2.9 | 3.3 | 150 | 0.9 | 62 |

The results showed that the recombinant B. licheniformis BLA1 constructed in Example 1 had high cell stability and low lysis degree during the cycle catalysis process of the reaction device. After 10 cycles, the cell retention rate was 96.7%, and the enzyme activity retention rate per unit cell was 97%, indicating the potential for industrial-scale production.

### Comparative Example 1

The gene encoding D-psicose-3-epimerase with the nucleotide sequence shown in SEQ ID NO. 7 was constructed into B. licheniformis using the same method as in Example 1 to obtain recombinant B. licheniformis BLA7, and D-psicose was produced using the whole-cell conversion method in Example 3. The results showed that at the same concentration, the conversion rate of recombinant B. licheniformis BLA7 was only 13.2%, and the conversion rate of recombinant B. licheniformis BLA1 was 2.3 times higher.

### Comparative Example 2

The gene encoding D-psicose-3-epimerase with the nucleotide sequence shown in SEQ ID NO. 8 was constructed into B. licheniformis using the same method as in Example 1 to obtain recombinant B. licheniformis BLA8, and D-psicose was produced using the whole-cell conversion method in Example 3. The results showed that at the same concentration, the conversion rate of recombinant B. licheniformis BLA8 was only 11.7%, and the conversion rate of recombinant B. licheniformis BLA1 was 2.6 times higher.

### Comparative Example 3

The gene encoding D-psicose-3-epimerase with the nucleotide sequence shown in SEQ ID NO. 9 was constructed into B. licheniformis using the same method as in Example 1 to obtain recombinant B. licheniformis BLAS, and D-psicose was produced using the whole-cell conversion method in Example 3. The results showed that at the same concentration, the conversion rate of recombinant B. licheniformis BLAS was only 8.3%, and the conversion rate of recombinant B. licheniformis BLA1 was 3.7 times higher.

Although the present disclosure has been disclosed with the above exemplary examples, it is not intended to limit the present disclosure. Any person skilled in the art can make various changes and modifications without departing from the spirit and scope of the present disclosure, so the scope of protection of the present disclosure shall be subject to the scope defined by the claims.

What is claimed is:

1. A recombinant Bacillus licheniformis, wherein B. licheniformis CICIM B1341 is used as a host and the recombinant B. licheniformis carries the gene set forth in SEQ ID NO:1.

2. The recombinant B. licheniformis according to claim 1, wherein pHY300-PLK is used as an expression vector.

3. The recombinant B. licheniformis according to claim 1, wherein the gene with nucleotide sequence set forth in SEQ ID NO:1 is integrated into a site of amylase encoding gene amyL in the B. licheniformis genome.

4. A whole-cell catalyst, comprising the recombinant B. licheniformis of claim 1.

5. A method for synthesis of D-psicose through whole-cell conversion, comprising using D-fructose as a substrate, and adding the recombinant B. licheniformis of claim 1 or a whole-cell catalyst comprising the recombinant B. licheniformis, reaction at 50-70° C. for 5-10 hours.

6. The method according to claim 5, wherein after the reaction is completed, the recombinant B. licheniformis or the whole-cell catalyst is recovered by centrifugation or filtration, reused, and added to a reaction system using the D-fructose as the substrate to synthesize D-psicose.


SEQUENCE LISTING (9 sequences)

SEQ ID NO: 1 - DNA, 882 bp, synthetic construct
  source 1..882: mol_type = other DNA
atgaaacatggcatctattatgcgtattgggaacaggaatgggaagcggattataaatattatatcgaaaaagtcgcgaaactgggctttgatatcctggaaatcgcggcgagcccgctgccgttttatagcgatatccagatcaatgaactgaaagcgtgcgcgcatggcaacggcatcacgctgacggtcggccatggcccgagcgcggaacagaatcttagcagccctgatccggatatccgcaaaaacgcgaaagcgttttatacggatctgctgaaacgcctgtataaactggatgtccatctgatcggcggcgcgctgtatagctattggccgatcgattatacgaaaacgatcgataaaaaaggcgattgggaaagaagcgtcgaaagcgtccgcgaagtcgccaaagtcgcggaagcgtgcggcgtcgatttttgccttgaagtcctgaaccgctttgaaaactatctgatcaacacggcgcaggaaggagtcgattttgtcaaacaggtcgatcataacaatgtcaaagtcatgctggatacgtttcatatgaacatcgaagaagatagcatcggcggcgcgatccgcacggcgggcagctatcttggccatctgcatacgggcgaatgcaaccgcaaagttccgggccgcggccgcatcccgtgggtcgaaatcggcgaagccctggccgatatcggctataacggcagcgtcgtcatggaaccgtttgtcagaatgggcggcacagtcggcagcaacatcaaagtctggcgcgatatcagcaacggcgcggatgaaaaaatgctggatcgcgaagcgcaggcggcgctggattttagccgctatgtcctggaatgccataaacatagctaa

SEQ ID NO: 2 - DNA, 267 bp, synthetic construct
  source 1..267: mol_type = other DNA
agtttatgtatagatattttcgaatatttaacttattggacacaataattttgaaatagggcattttgcacaagaaataatccaaaatagcccaaaaataagtttatgtatagatattttcgaatatttaacttattggacacaataattttgaaatagggcattttgcacaagaaataatccaaaatagcccaaaaataatccaacaattctaattattgttataataatgctgagctcccaaaattaattaagaggtgaaggaaa

SEQ ID NO: 3 - DNA, 133 bp, synthetic construct
  source 1..133: mol_type = other DNA
caggataagctccagatcctgctatcaataccaagtcactgaattacccgtcatgattcctttcctattgcttgttgttatgacgggtaacttctataattaggatttatttagagtgaatggttttttaccc

SEQ ID NO: 4 - DNA, 1539 bp, synthetic construct
  source 1..1539: mol_type = other DNA
atgaaacaacacaaacggctttatgcccgattgctgccgctgttatttgcgctcatcttcttgctgcctcactctgcagcagcggcggcaagtcttaatgggacgctgatgcagtattttgagtggtacatgccaaatgatggccaacattggaaacgcttacaaaatgactcggcatatttggctgaacacggtattactgccgtctggattcccccggcatataagggaacgagtcaagacgatgtaggctacggcgcttacgatctgtatgatttaggggagtttcatcaaaaagggacggttcggacaaagtacggcacaaagggagaactgcaatctgcgatcaacagtcttcattcccgggacatcaacgtttacggcgatgtagtcatcaaccacaaaggcggcgctgatgcgaccgaatatgtaacggctgttgaagtcgatcccgccgaccgcaaccgcgtaacatcaggagaacagcgaatcaaagcgtggacacattttcaattcccggggcgcggcagcacatacagcgatttcaaatggtattggtaccattttgacggaaccgattgggacgagtcccgaaagctgaaccgcatctataagtttcaaggaaaggcatgggattgggaagtttccaatgaaaacggcaactatgattacttgatgtatgccgacatcgattatgatcatcctgatgtcacggcagaaataaagagatggggaacgtggtatgccaatgagctgcaattggacggattccgccttgatgccgtcaaacacattaaattttcttttttgcgggattgggtcaatcatgtcagggaaaaaacagggaaggaaatgtttacggtagctgaatattggcagaatgacttaggtgcgctggaaaactatttgaacaaaacaaactttaatcattcagtgtttgacgtgccgcttcattaccagttccatgctgcatcgacacagggaggcggctatgatatgaggaaattgctgaacggaacagtcgtttccaagcatcctgtgaaagcggttacgtttgttgataaccatgatacacagccggggcaatcgcttgagtcgactgtccaaacatggtttaagccgctggcttacgcttttattttgacaagagaagcaggctacccgcagattttctacggggatatgtacgggacgaaaggagcctcgcagcgcgaaattcctgccctgaaacacaaaatcgaaccgatcttaaaagcgagaaaacaatatgcgtacggagcacagcatgattatttcgatcatcataacattgtcggctggacgagggaaggcgacagctcggttgcaaattctggtttggcggcgttaataacagacggacccggcgggacaaagcgaatgtatgtcggccggcaaaacgccggtgagacatggcatgacatcaccggaaaccgttccgattctgttgtcatcaatgcagaaggctggggagagtttcacgtaaacggcggatcggtttcgatctatgttcaaagatag

SEQ ID NO: 5 - DNA, 30 bp, synthetic construct
  source 1..30: mol_type = other DNA
gcgcggatccatgaagcacggtatctatta

SEQ ID NO: 6 - DNA, 30 bp, synthetic construct
  source 1..30: mol_type = other DNA
ccggctagctggagtgtttgtgacattcta

SEQ ID NO: 7 - DNA, 816 bp, synthetic construct
  source 1..816: mol_type = other DNA
atgatgcattcttgtgtaactattgccttagttcctcaaatcaagtcgggcccctggattttttgggaagacttagaagcgagtatggcccaagcgtctaggctgggatttgatgccatcgaactttttacagaagatgctaaaagtcttgataaagggctattggatggactattgtcaaaatattcattaaaactagcggcagtgggaagtggagctggaaaagttattcagggattaacgctgactgaccccaatccagaaatccgaacgaaagctatccattttattcaatccatgatggattttggtgcgcattttggggcggtgaccatcattggttccatgcaaggaaatgctttgcctgatcgtagagaggaatctttgatgcatttacaagaggccttggaatatctcgggaaacatgcggagaaactaggagtacctttgatttacgaacctttgaaccgttatgaaaccaatttatttaataccttgggttctggtaaagcatttgtagaaggtctatccacgcgtcatatcaaattactagctgatttgtttcacatgaacatagaagaaatagatatagctcaaagtatcctagaaaatggaccttggattggtcatgtgcatttagcagattccaatcgaagaccgatgggaatgggtcatacttccatgaaagaaatagcagcagctttacagcaaataaattaccaaggagccatatctgcggaagcatttccctggcctaatcctttggaagctgccgagcagaccatcaaaacataccaacaatattttaaaaaataa

SEQ ID NO: 8 - DNA, 912 bp, synthetic construct
  source 1..912: mol_type = other DNA
atggcattaaacgtaaaatttggcgtaagcacctggctctggacgtcccctttcgattccactacgctggcattatttcccaaaattaaatcgatgggttacgacgtagtagagattcccgtggaggataccagtttaatagacgctcccaagttgaaagaagcactggaggcaaatggtttggaaggtctaatttgcggtgcctttggtactacccgcgatttaacccacgaagacgaactctaccaacgtaacagcatcgaatacattacccaatgttttcagatttccgaagccattggttgttcttttgtatcgggtccgatgtattcagcggtgggtaaagccaggttagtttcgccggaacaaaagaaagttgaatgggaccgggcagttaccaacctgcaaaaagtgtgcgctatcgccgaccaatacggccaagccattgccttggaacccatcaatcgtttcgaaaccgatttaataaacaatgccgaagacgtgatgcagttaatccgcgacatcaatcatccggctgcccaggtttcgctggatggtttccacatgaacattgaagaaccagatatcgagaaagccattcttttaacgggcgataaactgcggcacgtgcaagtatccgaaaattaccgcggtacgccgggcaccggccaaacccgctgggatgccttttaccgcggcttaaccgccattaactaccagggcgccgttgccattgaaagtttcacccccaacgtaaaagaactggccggagccgtctgcatctggcgcccactcgccgaaagccaggatggttttgcttcggaaggcattcaatttttaaaaaaatgggctaaaggggaagaaaaggttaagcaggaagatggtattgcgagtgctttaatagcaaagtag

SEQ ID NO: 9 - DNA, 882 bp, synthetic construct
  source 1..882: mol_type = other DNA
atgaacccgatcggtatgcactacggtttctggtctcacaactgggatgaaatcgcgtacatcccgctgatggaaaaactggcatggctgggttttgatatctgcgaagttgcgtctgctgaatggggttattacgatgatgctcgtctgcgtgaactgaaagcttgcgctgatcataatggtctgggtattacctactctattggcctggaagcgaaatatgatctggcgtctgatgatccggctgttcgtgaaaacggcatccgtcacgttacccgtattctggaatctatgccaaaagttggtgcagctattctgaacggcgtgtcttacgcgggttggcaggcgttaccggatcacggcatcaccctggatgaaaaacgtcgtaaagaagaactggcgctggaatctatgagccgtctgatgaaagttgctgaagattgcggtgttctgtattgttgtgaagttgtgaaccgttttgaacagtatttactgaacaccgctaaagaaggtgttgaattcgttaaacgtctgggtagcccgaacgctcgtgttctgctggataccttccacatgaacatcgaagaagattctatggttgatgctattttagaagcgggcccgtggctgggtcatttccatgttggtgaaaacaaccgtcgtccggcgggtagcaccaaccgtctgccgtggaaagatatggcggcggctctgaaacaggttaactaccagggtgcgattgttatggaaccgttcgttctgatgggtggcaccattccgtatgatatcaaagtttggcgtgatctgtctggtggtgcgggtgaagcgggcctggatgaaatggcgggtcgtgcgtgtcgttttctgaaagaactgaccgcg